(19) United States
(12) Patent Application Publication
(10) Pub. No.: US 2009/0227838 A1
(43) Pub. Date: Sep. 10, 2009

(54) PROPELLABLE APPARATUS WITH PASSIVE SIZE CHANGING ABILITY

(75) Inventors: John J. Allen, Mendota Heights, MN (US); Randall A. Meyer, Edina, MN (US); Richard Cornelius, Wayzata, MN (US); Troy J. Ziegler, Plymouth, MN (US); Timothy P. Sheridan, Eagan, MN (US)

Correspondence Address: SCHWEGMAN, LUNDBERG & WOESSNER, P.A., P.O. BOX 2938, MINNEAPOLIS, MN, 55402, US

(73) Assignee: SoftScope Medical Technologies, Inc., Minnetonka, MN (US)

(21) Appl. No.: 12/401,424

(22) Filed: Mar. 10, 2009

Related U.S. Application Data
(60) Provisional application No. 61/068,864, filed on Mar. 10, 2008.

(51) Int. Cl. A61B 1/00

(52) U.S. Cl. 600/114

(57) ABSTRACT

An apparatus includes a self-enclosed tube, sized and shaped to fit within and engage a human or animal body cavity, the tube comprising an inner surface defining an enclosed region and an outer surface that turns outward to engage the body cavity and turns inward to encompass a central region defining a concentric longitudinal path, wherein the tube is powerable to provide relative movement of the tube relative to the cavity in at least one of a forward or reverse direction with respect to the longitudinal path, and a compressible structure, configured to bias the outer surface of the tube outward to engage the body cavity at a first outer diameter, the compressible structure being deformable inward in response to a compressive force to provide a second outer diameter that is less than the first outer diameter.

### CROSS-REFERENCE TO RELATED APPLICATIONS

This non-provisional application claims the benefit of priority under 35 U.S.C. §119(e) of U.S. Provisional Application Ser. No. 61/068,864, filed on Mar. 10, 2008, the specification of which is hereby incorporated by reference in its entirety.

### FIELD

This patent document relates generally to propellable apparatus, and more specifically, to a propellable apparatus with passive size changing ability.

### BACKGROUND

Endoscopes are routinely used in medical procedures to view the interior of a patient's body and to facilitate treatment of sites inside the body as atraumatically as possible. Some common types of endoscopes include: colonoscopes, such as to image or treat the colon, enteroscopes, such as for use in the stomach or small bowel, and bronchoscopes, such as for use in the trachea or bronchi. Other instruments can also be useful when inserted into a body lumen or cavity, either with or without an accompanying endoscope.

### OVERVIEW

One approach in facilitating use of an endoscope or other accessory includes providing an apparatus that can facilitate its introduction into or removal from a body lumen or cavity, such as described in Ziegler et al. U.S. Pat. No. 6,971,990, Ziegler et al. U.S. patent application Ser. No. 11/260,342 (which is published as U.S. Patent Application Publication No. 2006/0089533) and Ziegler et al. U.S. patent application Ser. No. 11/825,528, the disclosures of each of which are incorporated by reference herein in their entirety, including their description of a propellable apparatus and related methods.

For example, a drive structure can be mounted on the endoscope or other accessory. The drive structure can propel a self-enclosed toroidal membrane, such as to create propulsion force against the lumen or cavity wall. This can aid in advancing or withdrawing the endoscope or other accessory.

The present inventors have recognized, among other things, that there are several possible structures or methods that can be particularly advantageous such as when used to support this rotating toroidal membrane.

In some examples, an apparatus comprises a permeable or impermeable self-enclosed tube. The tube can be sized and shaped to fit within and engage a human or animal body cavity. The tube can comprise an inner surface defining an enclosed region and an outer surface that turns outward to engage the body cavity and turns inward to encompass a central region defining a concentric longitudinal path. An attachment can be coupled to the tube. The attachment can be configured to secure a payload. The tube can be powerable such as to provide relative movement of the tube relative to the cavity. This can help move the payload, with respect to the cavity, in at least one of a forward or reverse direction with respect to the longitudinal path. A compressible structure can be configured to bias the outer surface of the tube outward to engage the body cavity at a first outer diameter. The compressible structure can be deformable inward in response to a compressive force from a stricture in the body cavity to provide a second outer diameter that is less than the first outer diameter.

This overview is intended to provide an overview of subject matter of the present patent document. It is not intended to provide an exclusive or exhaustive explanation of the invention. The detailed description is included to provide further information about the present patent document.

### BRIEF DESCRIPTION OF THE DRAWINGS

FIG. 1 shows an example of a propellable apparatus, in accordance with one embodiment.

FIG. 2A shows a propellable apparatus within a body cavity, in accordance with one embodiment.

FIG. 2B shows the propellable apparatus of FIG. 2A within the body cavity, in accordance with one embodiment.

FIG. 2C shows the propellable apparatus of FIG. 2A within the body cavity, in accordance with one embodiment.

FIG. 3A shows a cross-section of a propellable apparatus, in accordance with one embodiment.

FIG. 3B shows a cross-section of a propellable apparatus, in accordance with one embodiment.

FIG. 3C shows a cross-section of a propellable apparatus, in accordance with one embodiment.

FIG. 3D shows a cross-section of a propellable apparatus, in accordance with one embodiment.

FIG. 4A shows an example of a compressible structure, in accordance with one embodiment.

FIG. 4B shows an example of a compressible structure, in accordance with one embodiment.

FIG. 5 shows another example of a compressible structure, in accordance with one embodiment.

FIG. 6 shows another example of the compressible structure of FIG. 5, in accordance with one embodiment.

FIG. 7 shows another example of a compressible structure, in accordance with one embodiment.

### DETAILED DESCRIPTION

A self-enclosed toroidal membrane can be used to create a propulsive force, such as between a propellable apparatus and the wall of the body cavity or lumen. The propellable apparatus can be used to help advance or maneuver an endoscope or other accessory, such as within the body cavity or lumen.

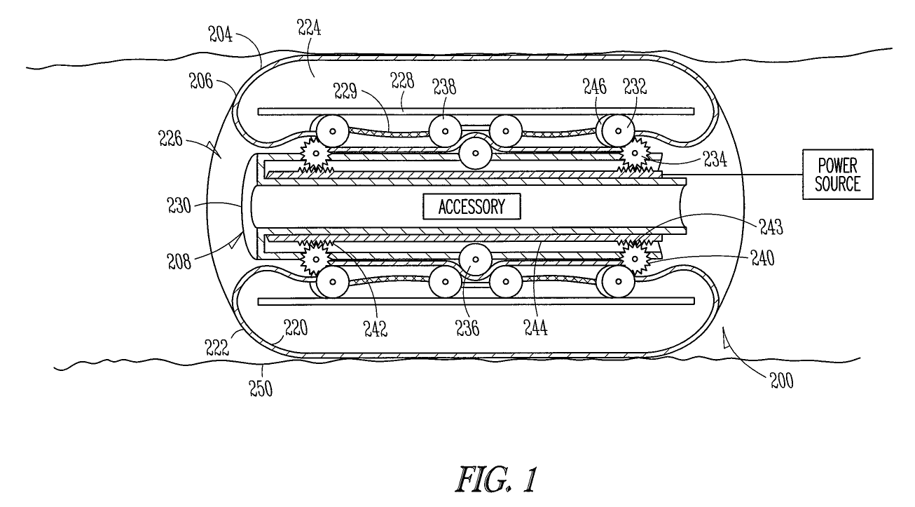

FIG. 1 shows a sectional view of an example of a propellable apparatus 200 that includes a toroidal self-enclosed tube 204. The self-enclosed tube 204 can be driven within the internal structure of the apparatus such as to create a propulsive force as its outer surface moves relative to the tissue wall 250. Self-enclosed tube 204 is generally toroidal or ring shaped. Self-enclosed tube 204 includes a flexible material 206. Flexible material 206 of self-enclosed tube 204 has an interior surface 220 and an exterior surface 222. Interior surface 220 of flexible material 206 defines an interior volume or enclosed region 224. Exterior surface 222 of flexible material 206 defines a central cavity 226.

The apparatus 200 also includes a frame 208. Frame 208 both supports and interacts with the flexible material 206 of the self-enclosed tube 204. Frame 208 includes a drive support structure 228 and a housing structure 230. Housing structure 230 is disposed in central cavity 226 defined by exterior surface 222 of flexible material 206 of self-enclosed tube 204. Drive support structure 228 is disposed within enclosed region 224 defined by interior surface 220 of flexible material 206 of self-enclosed tube 204.

In this example, drive support structure 228 and housing structure 230 each rotatably support a plurality of rollers. For example, a plurality of motive rollers 234 are shown contacting flexible material 206 of self-enclosed tube 204. Rotation of motive rollers 234 is capable of causing flexible material 206 to move relative to the rotational axis of each motive roller 234.

A worm gear 244 includes a first thread 242 and a second thread 243. The teeth 240 of a first set of motive roller 234 are shown mating with first thread 242 of worm gear 244. Accordingly, rotation of worm gear 244 will cause the first set of motive rollers 234 to rotate.

Housing structure 230 rotatably supports a plurality of stabilizing rollers 236. Each stabilizing roller 236 contacts the exterior surface 222 of flexible material 206 of self-enclosed tube 204. A plurality of suspended stabilizing rollers 238 are located proximate each stabilizing roller 236 and supported by spring-loaded supports 229 of drive support structure 228. Each suspended stabilizing roller 238 contacts interior surface 220 of flexible material 206 of self-enclosed tube 204. In some embodiments, suspended stabilizing roller 238 acts to bias exterior surface 222 of flexible material 206 against a stabilizing roller 236.

A suspended motive roller 232 is disposed proximate each motive roller 234. Each suspended motive roller 232 can be pivotally supported by drive support structure 228. In some embodiments, drive support structure 228 and suspended motive rollers 232 act to bias exterior surface 222 of flexible material 206 against motive rollers 234.

Various embodiments of housing structure 230 and drive support structure 228 are possible. One embodiment may be viewed as two tubes positioned with one inside the other. The outer tube being the drive support structure, which is located within the interior volume of the enclosed ring or bladder. The inner tube being the housing structure, which is located within the central cavity. In another embodiment, either the drive support structure, the housing structure or both may be comprised of a series of one or more beams that may or may not form the general shape of a cylinder.

The flexible material 206 of the self-enclosed tube 204 surface runs between the two tubes which are spaced in fixed relationship relative to each other. The distance between the two tubes is sufficient to accommodate the interlocking rollers or skids and to allow the flexible material 206 for self-enclosed tube 204 to pass between the support and housing structures even if the material folds over itself or is bunched up.

The present inventors have recognized that it is beneficial to creating a propulsive force for the outer surface of the flexible material 204 to be in close proximity to the tissue wall 250. In the case of a body cavity or body lumen, such as for example, the colon or small bowel, the present inventors have recognized that this propulsive force can increase as the diameter of the outer surface of the flexible material increases relative to the circumference of the body lumen. This increase in propulsive force may be driven by greater area of surface contact between the tissue wall 250 and the rotating toroidal surface of the device.

The propulsive force may also be increased due to increased contact pressure between these surfaces brought about by the increased diameter of the device. However, the present inventors have also recognized that having this relatively larger diameter for increasing propulsive force is at odds with a need to have as small a diameter as possible for introducing the apparatus (which can optionally be accompanied by an endoscope or other accessory) into the body lumen. Examples of orifices for introducing the endoscope and apparatus include the anal sphincter, or through the mouth and esophageal sphincter. In addition to these orifices and reduced-diameter sphincters, there can be other points of reduced lumen diameter, such as for example the iliocecal orifice between the small bowel and colon, or strictures in any of the body lumens such as brought on by scar tissue or growths such as cancers or polyps. These points of reduced diameter generally cannot accept introduction of rigid devices having diameters equal to the diameters of the internal lumens adjacent them without risk of injury or discomfort. The present inventors have also recognized that it will sometimes be desirable to have the device diameter variable such as to accommodate its extended use in one or more lumens of different diameters. An example of this would be for a device that is used to propel a scope in a retrograde approach through the colon and into the small bowel. The colon typically has a diameter that can be 50-100% greater than a diameter of the small bowel. It would be beneficial if the propulsive device can effectively propel the scope through the larger diameter colon and then on into the smaller diameter small bowel.

The present inventors have recognized that it is therefore desirable to have the outer diameter of the device that is configured to be variable, such as to allow a smaller diameter when passing through points or regions of reduced diameter, and to allow a larger drive diameter in larger diameter regions of the anatomy. The present inventors have also recognized that it is also desirable for this size transition to occur without the need for active actuation on the part of the operator. This is because it does not require an additional step in the procedure (and additional complexity of the device). This is also because these locations of reduced diameter may be unpredictable, for example, in where they are located, or in their degree of constriction.

One approach to providing some variability in diameter would be to use air or another compressible gas to inflate the flexible material. However, this approach may be limited in the range of diameters achievable because of the gas inflation pressure that would be needed. Other approaches to providing some variability can require operator actuation, which may be difficult, for example, to repeatedly actuate multiple times or to multiple different diameters using the same propulsion device during the same medical procedure.

By contrast, the present patent document describes, among other things, a variety of examples of one or more compressible structures such as can be used to help fill the volume of the diameter between (1) the outer diameter of the rigid drive mechanism that drives the toroidal flexible material; and (2) the desired outer diameter of the toroidal flexible material that provides propulsion in the body lumen. These compressible structures provide some at least partially solid structure beyond just pressurized gas used to inflate the toroidal flexible material, although the compressible structures can, in some examples, be used in combination with a pressurized gas that acts to expand the diameter of the toroidal surface.

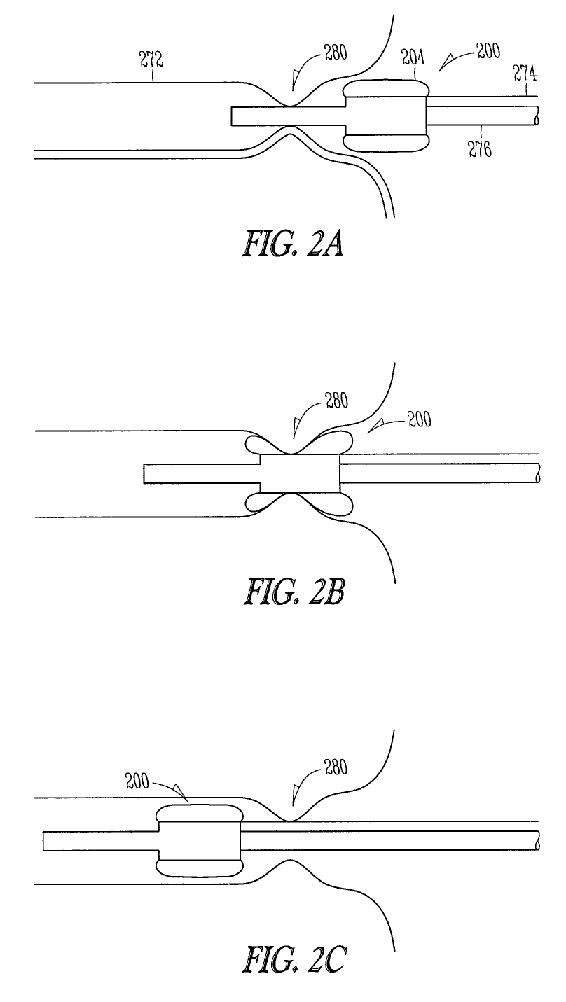

An illustrative example is shown in FIGS. 2A-2C, which show a propellable apparatus 200 within a body cavity 272, in accordance with one embodiment. Apparatus 200 includes a toroidal self-enclosed tube 204, which can be driven by a drive mechanism as discussed above, along with a drive cable 274. In this example, the apparatus 200 carries an endoscope or other accessory 276 within the body cavity.

Apparatus 200 includes a compressible structure configured to compress to a smaller diameter, when the device passes through a sphincter or other region of reduced diameter 280, such as shown in the example of FIG. 2B.

The apparatus 200 and compressible structure can then expand back out to its original diameter after passing through the region of reduced diameter 280, such as shown in the example of FIG. 2C.

In some examples, one or more of these compressible structures can be mounted to the outer surface of the hard drive mechanism, such that the flexible material 206 then slides over their outer surface(s) when the device drive is engaged to drive the flexible material.

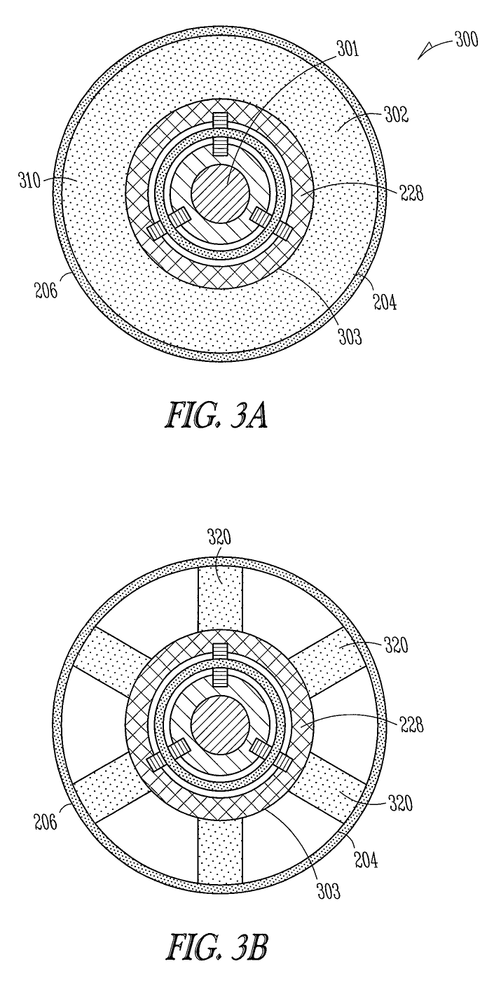

FIG. 3A shows a cross-section of a propellable apparatus 300 carrying an endoscope or other accessory 301, in accordance with one embodiment. In this example, the compressible structure includes a cellular or other foam material 310 that can be attached to the outer surface 303 of the rigid drive support structure 228 and is located within the enclosed region 302 of the propellable apparatus 300 between the outer surface 303 of the rigid drive support structure 228 and the outer surface of the flexible material 206 of the self-enclosed tube 204.

In one example in which the propellable apparatus is a device designed for use over an 11 millimeter diameter colonoscope, the rigid incompressible drive structure can have an outer diameter of about 22 millimeters. In this illustrative example, the foam then can be adhered to the outer surface of the rigid drive structure to yield an effective uncompressed outer diameter of the foam of about 33 millimeters. This foam can include a contiguous annular band of foam covering the whole outer surface of the drive structure and providing a somewhat cylindrical-like outer foam diameter, such as shown in the example of FIG. 3A. An example of a possible foam that can be used for this is Z60-I reticulated polyurethane foam, which is produced by Foamex International, of Linwood, Pa. In this example, the toroidal flexible material then traverses through the internal drive structure and wraps over the outer surface of the foam. In some examples, such foam compressible structure construction allows a 33 millimeter drive diameter, while compressing down to a diameter of 25 millimeters or less when passing through regions of reduced diameter.

FIGS. 3B-3D show cross-sections of other illustrative examples, in accordance with one or more embodiments. In these examples, a foam material on top of the drive structure can be configured in strips such as to only cover a fraction of the surface area while still similarly supporting the flexible material at an expanded outer diameter. Eliminating some of the mass of foam in this way can decrease the force needed to compress the foam to a smaller diameter as compared to the fully annular foam covering described above with respect to FIG. 3A.

For example, FIG. 3B shows a plurality of foam strips 320 attached longitudinally along the outer surface 303 of the rigid drive support structure 228 and located between the support structure and the outer surface of flexible material 206.

In FIG. 3C, the foam structure 330 includes a plurality of holes or spaces 322.

In FIG. 3D, the foam structure 340 includes a series of foam arches encircling the outer surface of the rigid drive support structure 228.

In the examples above, the thickness of the foam, the stiffness of the foam, and the percent area of the outer surface of the drive structure that is covered with foam can all be varied, such as to strike a desired balance between the desired compressed diameter for passing through a restricted diameter, the desired compressive force for compressing the foam, and the desired expansive force of the foam for providing the propulsive force at the toroidal flexible material when the foam is in a partially or fully expanded state within the body lumen. The diameters cited above are provided by way of illustrative example, and not by way of limitation. Other sizes of endoscopes or other accessories, and other diameters of the rigid drive structure of a propellable apparatus can be used as desired, such as for different anatomies, while still using and benefiting from a passively expandable and passively compressible structure that does not require user or other actuation to conform to different anatomical sizes in use.

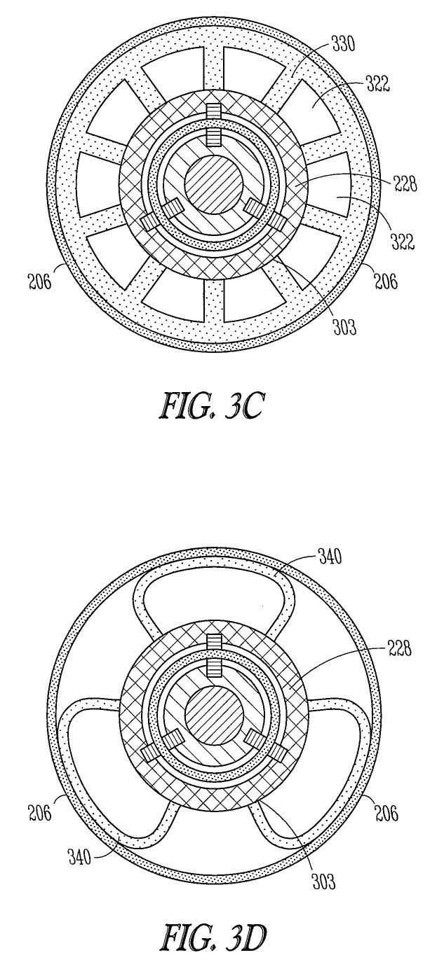

FIGS. 4A and 4B show an example of a compressible structure 402, in accordance with one embodiment. In this example, the compressible structure includes multiple bowed elements 404. A bowed element 404 can be connected to the outer surface of the drive support structure 228 at one end 406. At the other end, the bowed element 404 can be connected to the outer surface of the drive support structure 228 using a slide track mechanism 408. In some examples, the bowed elements 404 can be used to act as leaf springs, which are bowed out to support the flexible material of the self-enclosed tube, but which are able to compress as the one end moves along the slide track 408 when an external compressive force on the flexible material increases. In some examples, these bowed elements 404 can be made of stainless steel, nitinol, or one or more of any number of engineering polymers. The non-sliding connection 406 of the bowed element 404 to the outer surface of the drive support structure 228 can include a pinned connection. This can allow rotation of that end of the bowed element 404 as the other end moves in the slide 408 on the other end when the bowed element 404 is compressed down towards the outer surface of the drive support structure 228.

As shown in FIG. 4B, bowed elements 404 can be arranged on the outer surface of the drive support structure 228 to provide an overall compressible structure for a propellable apparatus.

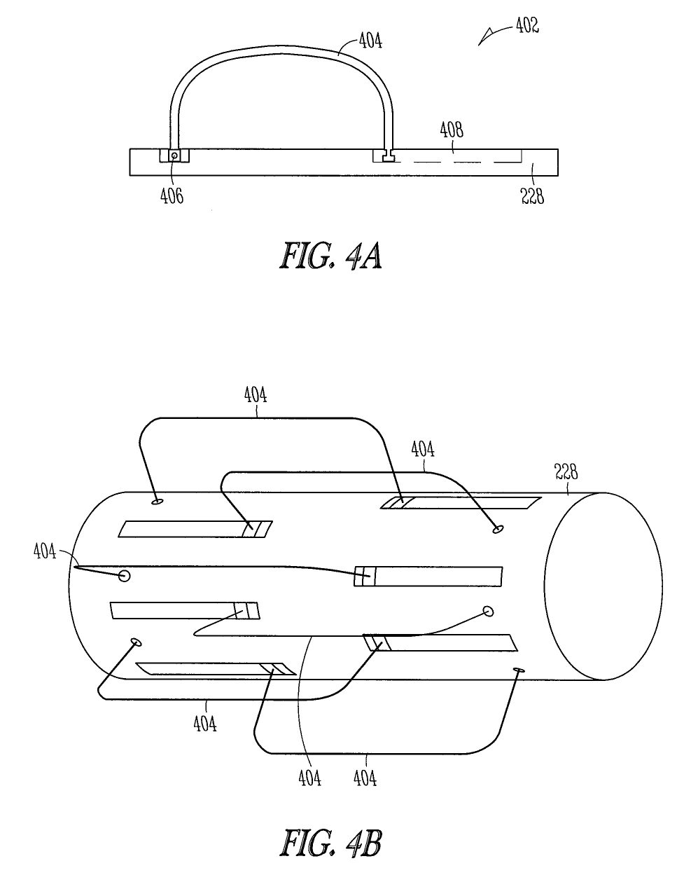

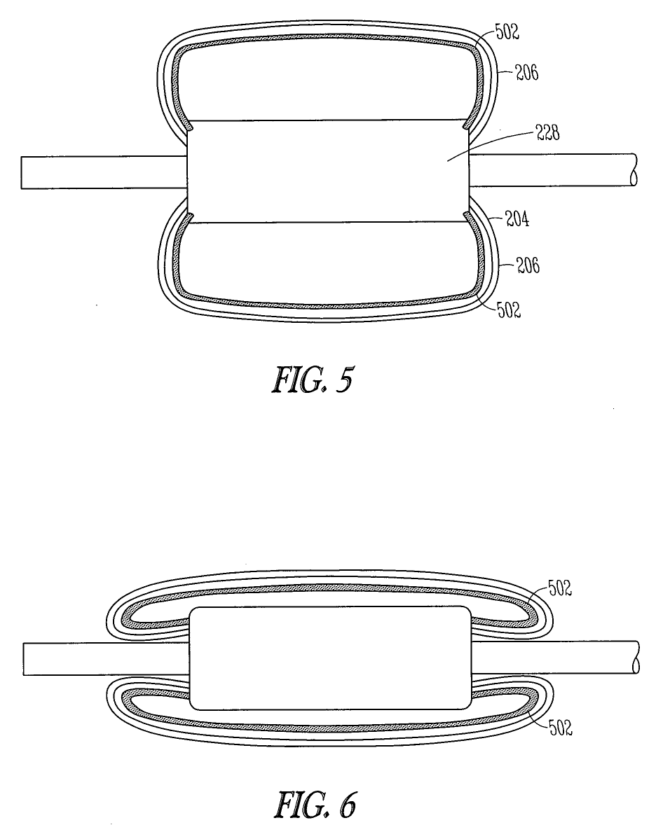

FIGS. 5 and 6 show another example of a compressible structure, in accordance with one embodiment. In this example, the compressible structure includes multiple bowed strut elements 502 supporting the flexible material 206 of self-enclosed tube 204. The ends of the elements 502 can be attached at or near opposite ends of the drive support structure 228. These connections can be fixed, pinned (such as to allow rotation), or in respective slide track mechanisms (such as to allow a degree of axial movement).

In this example, the regions of maximum strain upon the bowed elements 502 when compressed to a reduced diameter state can be located longitudinally outward from the ends of the cylindrical-like rigid drive structure rather than radially outward from its cylindrical-like circumference. This can permit more space for the deformation of the bowed elements, because the space taken up by the drive support structure 228 over the endoscope or other accessory is available for flexing and attached portions of the bowed elements in front of or behind the ends of the cylindrical-like drive structure, such as shown in FIG. 6.

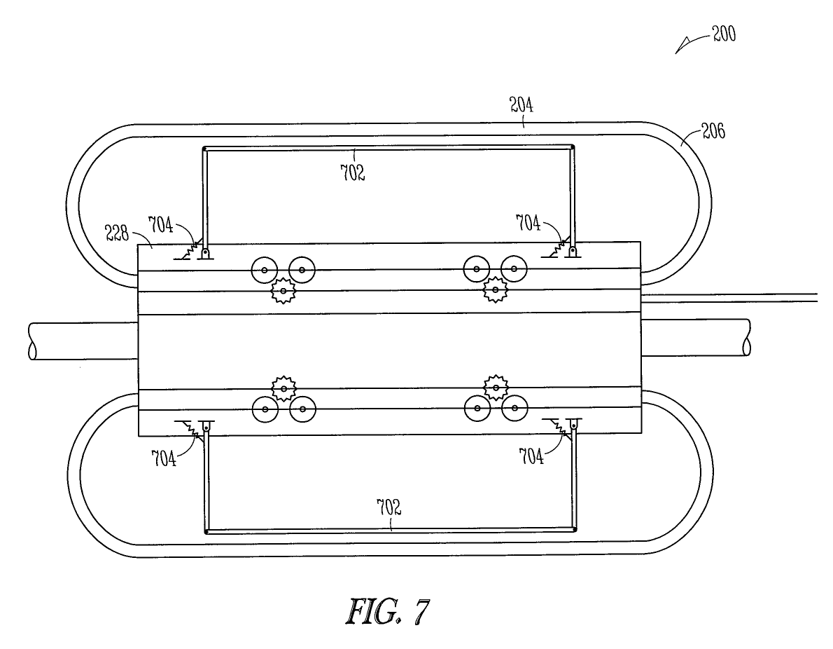

FIG. 7 shows another example of a compressible structure, in accordance with one embodiment. In this example, the compressible structure can include multiple linkages of struts 702. Such linkages 702 can be spring loaded 704 such as at the anchor points at the drive support structure 228. This can permit the linkages 702 to support the toroidal flexible material 206 at its larger diameter, and to collapse down to a reduced diameter with increased compressive force on the toroidal flexible material 206. In some examples, the struts 702 making up a linkage can be made of stainless steel wires or members, such as with holes on the ends such as to enable pinning the struts together at the joints of the linkage.

These disclosed examples show a number of possible structures that can achieve, without requiring user-actuation, a desired variable toroidal flexible material diameter for the propellable apparatus, which can be used to propel or maneuver an endoscope or other accessory. Other materials or variations of these examples can be used, such as to achieve a similar desired variable diameter, which can benefit the performance of the propulsion system.

### Additional Notes

The above Detailed Description includes references to the accompanying drawings, which form a part of the Detailed Description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a self-enclosed tube, sized and shaped to fit within and engage a human or animal body cavity, the tube comprising an inner surface defining an enclosed region and an outer surface that turns outward to engage the body cavity and turns inward to encompass a central region defining a concentric longitudinal path, wherein the tube is powerable to provide relative movement of the tube relative to the cavity in at least one of a forward or reverse direction with respect to the longitudinal path; and
a compressible structure, configured to bias the outer surface of the tube outward to engage the body cavity at a first outer diameter, the compressible structure being deformable inward in response to a compressive force to provide a second outer diameter that is less than the first outer diameter.

2. The apparatus of claim 1, wherein the compressible structure includes foam material located within the enclosed region.

3. The apparatus of claim 1, wherein the compressible structure includes a plurality of bowed members located within the enclosed region.

4. The apparatus of claim 1, wherein the compressible structure includes a plurality of bowed strut members located within the enclosed region.

5. The apparatus of claim 1, wherein the compressible structure includes a plurality of spring-loaded linked struts located within the enclosed region.

6. The apparatus of claim 1, further including a frame comprising a support structure located within the enclosed region and a housing structure located within a central cavity of the self-enclosed tube.

7. The apparatus of claim 6, wherein the compressible structure includes foam material attached to the support structure.

8. An apparatus comprising:
a self-enclosed toroidal tube, sized and shaped to fit within and engage a human or animal body cavity, the tube comprising a flexible material having an inner surface defining an enclosed region and an outer surface that turns outward to engage the body cavity and turns inward to encompass a central region defining a concentric longitudinal path;
an attachment coupled to the tube, the attachment to secure a payload, wherein the tube is powerable to provide relative movement of the tube relative to the cavity, and to thereby help to move the payload with respect to the cavity, in at least one of a forward or reverse direction with respect to the longitudinal path;
a frame including a drive support structure located within the enclosed region and a housing structure located within a central cavity of the self-enclosed tube; and
a compressible support structure coupled to the drive support structure and configured to bias the outer surface of the tube outward to engage the body cavity at a first outer diameter, the compressible structure being deformable inward in response to a compressive force to provide a second outer diameter that is less than the first outer diameter.

9. The apparatus of claim 8, wherein the compressible structure includes foam material located within the enclosed region between the support structure and the flexible material.

10. The apparatus of claim 9, wherein the foam includes a plurality of foam strips longitudinally extending along an outer surface of the support structure.

11. The apparatus of claim 8, wherein the compressible structure includes a plurality of bowed members located within the enclosed region.

12. The apparatus of claim 8, wherein the compressible structure includes a plurality of bowed strut members located within the enclosed region.

13. The apparatus of claim 8, wherein the compressible structure includes a plurality of spring-loaded linked struts located within the enclosed region.

14. A method comprising:
deploying a propellable self-enclosed tube within a cavity;
decreasing a diameter of the self-enclosed tube to a first diameter when a compressive force occurs within the cavity; and
passively expanding the diameter of the self-enclosed tube to a second diameter, larger than the first diameter, when the compressive force is passed.

15. The method of claim 14, wherein decreasing a diameter and passively expanding the diameter include providing a compressible structure within the self-enclosed tube, the compressible structure being configured to bias an outer surface of the tube outward to engage a wall of the cavity at the second diameter, the compressible structure being deformable inward in response to the compressive force to provide the first diameter.

16. The method of claim 15, wherein the compressible structure includes a foam material.

17. The method of claim 15, wherein the compressible structure includes a plurality of bowed members.

18. The method of claim 15, wherein the compressible structure includes a plurality of bowed strut members.

19. The method of claim 15, wherein the compressible structure includes a plurality of spring-loaded linked struts.

20. The method of claim 14, further including securing a payload to the self-enclosed tube for transport within the cavity.
